(19) United States
(12) Patent Application Publication
(10) Pub. No.: US 2022/0220527 A1
(43) Pub. Date: Jul. 14, 2022

(54) METHOD FOR MEASURING THE ACTIVITY OF A CULTURE OF MICROALGAE

(71) Applicant: TotalEnergies SE, Courbevoie (FR)

(72) Inventors: Bertrand Segues, Pau (FR); Cecilia Sambusiti, Pau (FR); Patrick Baldoni-Andrey, Pau (FR)

(21) Appl. No.: 17/594,599

(22) PCT Filed: Apr. 25, 2019
(86) PCT No.: PCT/IB2019/000586

§ 371 Date: Oct. 22, 2021

(51) Int. Cl. C12Q 1/02; C12M 1/00; C12M 1/34

(52) U.S. Cl. CPC C12Q 1/02; C12M 21/02; C12M 41/06; C12M 41/32; C12M 23/58

(57) ABSTRACT

The invention relates to a method for characterizing the activity of a culture comprising microalgae, the method comprising the steps of: supplying a sample of the culture from a bioreactor to a chamber fluidically connected to the bioreactor; illuminating the sample in the chamber; measuring the concentration of dissolved oxygen in the sample in order to determine a rate of oxygen production by the illuminated sample. The invention also relates to a device and an apparatus for characterizing a culture comprising microalgae.

### TECHNICAL FIELD

The present invention relates to a method and a device for measuring the activity and growth of a culture comprising microalgae and optionally bacteria.

### TECHNICAL BACKGROUND

The use of microalgae within a biological wastewater treatment has gained an increasing interest over the years. More particularly, microalgae are able to uptake nutrients such as ammonium, nitrate and phosphate in order to produce biofuels, or value-added products. Furthermore, microalgae can capture carbon dioxide (CO₂) in order to produce oxygen and carbon (carbohydrates, algae cells, lipids . . . ) due to their photosynthetic activity. The produced oxygen may be used by bacteria present in the wastewater to oxidize ammonium into nitrate.

The monitoring of the microalgae activity and growth, notably in the presence of bacteria, makes it possible to determine the kinetic and stoichiometric parameters essential for describing the behavior of biological processes, the optimal conditions for their growth so as to optimize biological wastewater treatment. Moreover, when bacteria are present in wastewater, the study of their activity could represent an ideal solution for understanding the interactions between microbial populations and microalgae as a function of environmental parameters. This allows to obtain information in order to predict the performances and the efficiency of these treatments.

The article of S. Rossi et al. (Activity assessment of microalgal-bacteria consortia based on respirometric tests), 2018 (doi: 10.2166/wst.2018.078) describes an experimental respirometric protocol to determine oxygen uptake rates and oxygen production rates by a microalgae-bacteria consortium at laboratory scale.

The article of G. Vargas et al. (Assessment of microalgae and nitrifiers activity in a consortium in a continuous operation and the effect of oxygen depletion), 2016 (doi: 10.1016/j.ejbt.2106.08.002) concerns a consortium of nitrifying bacteria and microalgae capable of operating with low requirements of dissolved oxygen, using aerobic sludge from wastewater treatment plants. According to this article, a methodology for measuring specific activities of nitrifying bacteria and microalgae was established by comparing the rates in variation of inorganic nitrogen compounds. The method of this article is carried out in a discontinuous manner and at laboratory scale.

The article of A. M. J. Kliphuis et al. (Light respiration in Chlorella sorokiniana), 2011 (doi: 10.1007/s10811-010-9614-7) describes the immediate post-illumination respiratory 02 uptake rate of a microalgal culture in situ, using fiber-optic oxygen microsensors and a small and simple extension of the cultivation system. This method allows rapid and frequent measurements without disturbing the cultivation and growth of the microalgae.

The article of A. Ruiz-Martinez et al. (Behavior of mixed Chlorophyceae cultures under prolonged dark exposure. Respiration rate modeling) concerns the study of the behavior of three different microalgal cultures, when exposed for a long period to dark conditions, based on respirometry.

Document US 2012/015671 1 describes a system and a method that provide the joined performance of optoelectric and respirometric sensors for instant and accurate ascertainment of biochemical oxygen demand in liquid industrial wastes.

Document U.S. Pat. No. 5,811,255 describes an apparatus and a method for anaerobic and aerobic respirometry. The apparatus and the method provide for automatically collecting and analyzing the data required to calibrate mathematical models for bioprocesses that involve anaerobic respiration and dehalogenation. Document U.S. Pat. No. 6,905,872 describes an on-line respirometer and a method for using the respirometer in order to determine the oxygen uptake of respiring samples such as a slurry sample comprising a mixture of sewage and an aqueous culture of microorganisms.

Document U.S. Pat. No. 4,314,969 describes a submersible recording respirometer including an oxygen analyzer which measures the concentration of oxygen absorbed by a wastewater sample contained in an aeration chamber.

There is still a need for a method for characterizing a culture comprising microalgae and optionally bacteria, in a quick, online, automated and efficient way, notably by measuring the concentration of dissolved oxygen (and optionally the concentration of dissolved carbon dioxide), in order to be able to assess and monitor the growth of the culture, and also in order to determine the respective activity of microalgae and bacteria in the culture, when bacteria are present.

### SUMMARY OF THE INVENTION

It is a first object of the invention to provide a method for characterizing the activity of a culture comprising microalgae, the method comprising the steps of:
supplying a sample of the culture from a bioreactor to a chamber fluidically connected to the bioreactor;
illuminating the sample in the chamber;
measuring the concentration of dissolved oxygen in the sample in order to determine a rate of oxygen production by the illuminated sample.

According to some embodiments, the method further comprises the steps of:
keeping a sample of the culture from the bioreactor in the absence of light;
measuring the concentration of dissolved oxygen in said sample in order to determine a rate of oxygen consumption by the sample in the absence of light.

According to some embodiments, the sample kept in the absence of light is the same as the sample which is illuminated.

According to some embodiments, the sample kept in the absence of light is different from the sample which is illuminated.

According to some embodiments, the step of keeping a sample in the absence of light is carried out in the chamber.

According to some embodiments, the step of keeping a sample in the absence of light is carried out in an additional chamber fluidically connected to the chamber and/or the bioreactor, the sample being preferably supplied from the chamber to the additional chamber, or directly from the bioreactor to the additional chamber.

According to some embodiments, the rate of oxygen production by microalgal photosynthesis is determined by adding the rate of oxygen production of the illuminated sample and the rate of oxygen consumption of the sample in the absence of light.

According to some embodiments, the concentration of dissolved oxygen in the sample is measured during the step of illuminating the sample and/or during the step of keeping a sample in the absence of light.

According to some embodiments, the step of illuminating the sample is carried out for a first duration and/or the step of keeping a sample in the absence of light is carried out for a second duration, and the rate of oxygen production by the illuminated sample is determined by calculating the derivative of the concentration of dissolved oxygen measured during the step of illuminating the sample as a function of time and/or the rate of oxygen consumption by the sample kept in the absence of light is determined by calculating the derivative of the concentration of dissolved oxygen measured during the step of keeping a sample in the absence of light as a function of time.

According to some embodiments, the rate of oxygen production by the illuminated sample is determined by measuring the concentration of dissolved oxygen in the sample before and after the step of illuminating the sample, and/or the rate of oxygen consumption by the sample in the absence of light is determined by measuring the concentration of dissolved oxygen in the sample before and after the step of keeping the sample in the absence of light.

According to some embodiments, more than one steps of measuring the concentration of dissolved oxygen are carried out, and after a first step of measuring the concentration of dissolved oxygen in the sample, the method comprises a step of renewing the sample in the chamber with a new sample from the bioreactor and a second step of measuring the concentration of dissolved oxygen in the sample.

According to some embodiments, the first step of measuring the concentration of dissolved oxygen in the sample is carried out during the step of illuminating the sample and the second step of measuring the concentration of dissolved oxygen in the sample is carried out during the step of keeping the sample in the absence of light; or the first step of measuring the concentration of dissolved oxygen in the sample is carried out during the step of keeping the sample in the absence of light and the second step of measuring the concentration of dissolved oxygen in the sample is carried out during the step of illuminating the sample; or the first and the second steps of measuring the concentration of dissolved oxygen in the sample are carried out during the step of illuminating the sample; or the first and the second steps of measuring the concentration of dissolved oxygen in the sample are carried out during the step of keeping the sample in the absence of light.

According to some embodiments, the method comprises more than one steps of illuminating samples, and more than one steps of keeping samples in the absence of light, each step of illuminating a sample alternating with a step of keeping a sample in the absence of light, the method preferably comprising measuring the concentration of dissolved oxygen during the steps of illuminating the samples, or during the steps of keeping the samples in the absence of light, or during both the steps of illuminating the samples and the steps of keeping the samples in the absence of light.

According to some embodiments, the culture also comprises bacteria.

According to some embodiments, at least one substrate for bacteria is added into the sample during the step of illuminating the sample and/or during the step of keeping the sample in the absence of light.

According to some embodiments, at least one inhibitor or toxic compound for bacteria or microalgae is added into the sample during the step of illuminating the sample and/or during the step of keeping the sample in the absence of light.

According to some embodiments, when bacteria are present in the culture: the rate of oxygen consumption by bacteria is determined by carrying out a first step of illuminating a sample and measuring the concentration of dissolved oxygen in order to determine a first rate of oxygen production by the illuminated sample, said sample being devoid of inhibitors or toxic compounds for bacteria, and carrying out a second step of illuminating a sample and measuring the concentration of dissolved oxygen in order to determine a second rate of oxygen production by the illuminated sample, said sample comprising at least one inhibitor or toxic compound for bacteria, and by calculating the difference between the second and the first rates of oxygen production; and/or the rate of oxygen consumption by bacteria is determined by carrying out a first step of keeping a sample in the absence of light and measuring the concentration of dissolved oxygen in order to determine a third rate of oxygen consumption by the sample in the absence of light, said sample being devoid of inhibitors or toxic compounds for bacteria, and carrying out a second step of keeping a sample in the absence of light and measuring the concentration of dissolved oxygen in order to determine a fourth rate of oxygen consumption by the sample in the absence of light, said sample comprising at least one inhibitor or toxic compound for bacteria, and by calculating the difference between the third and the fourth rates of oxygen consumption.

According to some embodiments, the method comprises a step of measuring dissolved carbon dioxide concentration in the sample, in order to determine a rate of carbon dioxide consumption or production by the illuminated sample and/or the sample kept in the absence of light.

According to some embodiments, the microalgae are chosen from chlorophyceae, xanthophyceae, chrysophyceae, bacillariophycea, cryptophyceae, dinophyceae, chloromonadineae, euglenineae, phaeophyceae, rhodophyceae, cyanophyceae.

According to some embodiments, the culture comprises an aqueous medium deriving from produced water, fresh water, sea water, aquifer water, or waste water.

According to some embodiments, the supply of sample from the bioreactor to the chamber is carried out in a continuous manner.

According to some embodiments, the supply of sample from the bioreactor to the chamber is carried out in a discontinuous manner.

According to some embodiments, the method further comprises a step of measuring the pH of the sample.

According to some embodiments, the method further comprises a step of measuring the turbidity of the sample.

Another object of the invention is to provide a device for characterizing a culture comprising microalgae, the device comprising:
a bioreactor configured to receive a culture comprising microalgae;
a chamber having an inlet and an outlet, and fluidically connected to the bioreactor so that it can be directly supplied via the inlet with a sample of culture from the bioreactor;
a source for emitting light in the chamber;
at least one sensor for measuring the concentration of dissolved oxygen in the sample.

Another object of the invention is to provide an apparatus for characterizing a culture comprising microalgae, the apparatus comprising:
a chamber having an inlet and an outlet, the chamber being configured to be fluidically connected to a bioreactor configured to receive a culture comprising microalgae, so that the chamber can be directly supplied via the inlet with a sample of culture from the bioreactor;
a source for emitting light in the chamber;
at least one sensor for measuring the concentration of dissolved oxygen in the sample.

According to some embodiments, the chamber is sealed.

According to some embodiments, the device or apparatus comprises at least one additional chamber having an inlet and an outlet, the additional chamber being fluidically connected to the chamber and the bioreactor.

According to some embodiments, the device or apparatus comprises a sensor for measuring the concentration of dissolved oxygen located in the chamber and/or comprises a sensor for measuring the concentration of dissolved oxygen located in the additional chamber.

According to some embodiments, the device or apparatus comprises a sensor for measuring the concentration of dissolved oxygen upstream of the inlet and/or downstream of the outlet of the chamber; and/or comprises a sensor for measuring the concentration of dissolved oxygen upstream of the inlet and/or downstream of the outlet of the additional chamber.

According to some embodiments, the additional chamber is devoid of a source for emitting light.

According to some embodiments, the device or apparatus comprises at least one sensor for measuring the concentration of dissolved carbon dioxide in the sample.

According to some embodiments, the device or apparatus comprises one or more sensors for measuring the pH of the sample, the temperature of the sample, and/or the turbidity of the sample.

According to some embodiments, the device or apparatus comprises at least one pump for the circulation of the sample from the bioreactor to the chamber and/or from the chamber to the bioreactor.

According to some embodiments, the device or apparatus comprises one or more injectors for adding a substrate for microalgae and/or bacteria or an inhibitor or toxic compound for bacteria or microalgae into or upstream of the chamber and/or into or upstream of the additional chamber.

Another object of the invention is to provide a computer program comprising instructions for performing the method described above.

Another object of the invention is to provide a data storage medium having recorded thereon the computer program described above.

Another object of the invention is to provide a system comprising a processor coupled to a memory having recorded thereon the computer program described above.

Another object of the invention is to provide an assembly comprising the system and the apparatus or device described above.

The present invention makes it possible to address the need mentioned above. In particular, the invention provides a method for characterizing a culture comprising microalgae and optionally bacteria, in a quick, online, automated and efficient way, notably by measuring the concentration of dissolved oxygen (and optionally the concentration of dissolved carbon dioxide), in order to be able to assess and monitor the growth of the culture, and also in order to determine the respective activity of microalgae and bacteria in the culture, when bacteria are present.

This is achieved by a method which allows to directly supply a sample located in a bioreactor to a chamber fluidically connected to the bioreactor, in order to perform the measurement of dissolved oxygen and optionally the measurement of dissolved carbon dioxide. Advantageously, this supply may be carried out in a continuous way in order to increase the rapidity of the process.

Due to the illumination of the sample in the chamber, the method according to the invention makes it possible to assess the photosynthetic activity of the microalgae and therefore their oxygen production as well as the amount of oxygen consumed for the respiration of microalgae and optionally for the respiration of bacteria. Advantageously, due to the step of keeping the sample in the absence of light, the method according to the invention makes it possible to measure oxygen consumption related to the respiration of microalgae and optionally the respiration of bacteria. Still advantageously, when bacteria are present, the injection of a substrate such as a nutrient or a carbon source and/or inhibitors and/or toxic substances for the bacteria makes it possible to assess the respective activity of microalgae and bacteria. For example, during a step of keeping the sample in the absence of light, the injection of a substrate for the bacteria allows to measure their maximal activity. Or, during the step of keeping the sample in the absence of light, the injection of inhibitors or toxic substances for bacteria makes it possible to decrease or inhibit their activity in order to measure the consumption of oxygen due to the respiration of the microalgae. Therefore, such method allows to assess the behavior of microorganisms (both microalgae and bacteria) in the presence of inhibitors or toxic substances.

In addition, the presence of a chamber comprising a source of light and the presence of an additional chamber devoid of a source of light, as well as the fact that the chamber and the additional chamber are fluidically connected to the bioreactor, allow the automatization of the process and enhance its rapidity.

Still advantageously, the method also allows the measurement of other parameters important for the monitoring of the growth and activity of microalgae and/or bacteria such as carbon dioxide, pH, turbidity or optical density. For example, during the illumination of the sample in the chamber, the consumption of carbon dioxide can be measured, this amount corresponding to the amount of carbon dioxide used by the microalgae for the photosynthesis as well as to the amount of carbon dioxide produced by microalgae and optionally bacteria for their respiration.

### BRIEF DESCRIPTION OF THE DRAWINGS

FIG. 1 schematically illustrates the device according to one embodiment of the present invention.

FIG. 2 schematically illustrates the device according to another embodiment of the present invention.

FIG. 3 illustrates the concentration of dissolved oxygen (A) and the dissolved oxygen production rate (B) in a sample of a culture comprising microalgae as a function of time. The concentration of dissolved oxygen (mg/L) and the oxygen production rate (mg/L.h) can be read on the Y-axis and time (h) can be read on the X-axis.

FIG. 4 illustrates the concentration of dissolved oxygen (A) and the dissolved oxygen production rate (B) in a sample of a culture comprising microalgae and bacteria as a function of time. The concentration of dissolved oxygen (mg/L) and the oxygen production rate (mg/L.h) can be read on the Y-axis and time (h) can be read on the X-axis.

### DESCRIPTION OF EMBODIMENTS

The invention will now be described in more detail without limitation in the following description.

### Microalgal Culture

The culture according to the invention comprises microalgae. By “microalgae” is meant microscopic algae, the maximum dimension of which can range from a few micrometers (μm) to a few hundred micrometers. Microalgae are capable of performing photosynthesis in the presence of light by transforming carbon dioxide into carbohydrates and producing oxygen. Simultaneously (in the presence of light), microalgae consume oxygen for their respiration. However, in the presence of light the amount of oxygen consumed is negligible compared to the produced oxygen due to the photosynthesis. Microalgae also consume oxygen in the absence of light.

Microalgae can be native of an environment or selected by using an official culture collection. Microalgae can be for example one of the types of chlorophyceae, xanthophyceae, chrysophyceae, bacillariophycea, cryptophyceae, dinophyceae, chloromonadineae, euglenineae, phaeophyceae, rhodophyceae, cyanophyceae.

According to some embodiments, the culture may comprise only one strain of microalgae.

According to other embodiments, the culture may comprise more than one strains of microalgae, for example 2, or 3, or 4, or 5, or 10 or more than 10 different strains of microalgae.

Preferably, the microalgae according to the invention are autotrophs, which means that they consume inorganic carbon sources to produce organic substances.

Alternatively, the microalgae according to the invention are mixotrophs. A “mixotroph” is an organism that can use a mix of different sources of carbon (organic and inorganic carbon), instead of having a single trophic mode on the continuum from complete autotrophy at one end to heterotrophy at the other.

The microalgae may have a concentration in the culture from 0.1 to 5 g/L, and preferably from 0.3 to 2 g/L. This concentration can be measured by measuring the total solids concentration or the turbidity or the optical density of the sample at 680 nm. Alternatively, this concentration can be measured by cell counting.

The culture may comprise an aqueous medium wherein the microalgae are cultivated. This aqueous medium may derive from produced water (deriving from the extraction of hydrocarbons from a subterranean formation), fresh water, sea water, aquifer water, or wastewater.

The culture may have a concentration of ammonium cation from 0 to 200 mg/L

The culture may have a concentration of phosphate anions from 3 to 150 mg/L

The culture may have a concentration of nitrate anions from 0 to 1500 mg/L

The culture may have a concentration of dissolved organic carbon from 0 to 500 mg/L

The culture may have a turbidity from 0 to 200 NTU

The culture may have a concentration of dissolved oxygen from 1 to 21 mg/L

The culture may have a pH from 2 to 11

The culture may have a salinity from 0 up to 300 g/L

The culture may have a temperature from 0 to 40° C.

According to some preferred embodiments, the culture may comprise bacteria.

According to some embodiments, the bacteria present in the culture are autotrophic bacteria. When autotrophic bacteria are present in the culture, it is preferable that they are ammonia oxidizing bacteria (bacteria oxidizing ammonia to nitrite) and nitrite-oxidizing bacteria (bacteria oxidizing nitrite to nitrate) Such bacteria can be native in the aqueous medium

According to other embodiments, the bacteria present in the culture are heterotrophic bacteria. Such bacteria can be native in the aqueous medium.

According to preferred embodiments, the culture comprises both autotrophic and heterotrophic bacteria.

The bacteria may be in a concentration in the culture from 0 to 0.5 g/L, and preferably from 0 to 0.2 g/L. This concentration can be measured by measuring the total solids concentration of the sample.

In some embodiments the ratio of bacteria concentration to microalgae concentration in the culture may be equal to or lower than 20% and preferably lower than 10%.

In the culture, bacteria (if present) consume oxygen due to their respiration. The presence of substrates (such as a carbon source or a nutrient) in the culture may increase the activity of the bacteria, therefore increasing their oxygen consumption. The presence of inhibitors or toxic substances may decrease or inhibit the activity of the bacteria and therefore decrease or suppress their oxygen consumption.

Device for characterizing the activity of a culture comprising microalgae The invention relates to a device for characterizing a culture comprising microalgae and notably for measuring the dissolved oxygen production and consumption rate in a sample of a culture comprising microalgae and optionally bacteria, the culture being as described above.

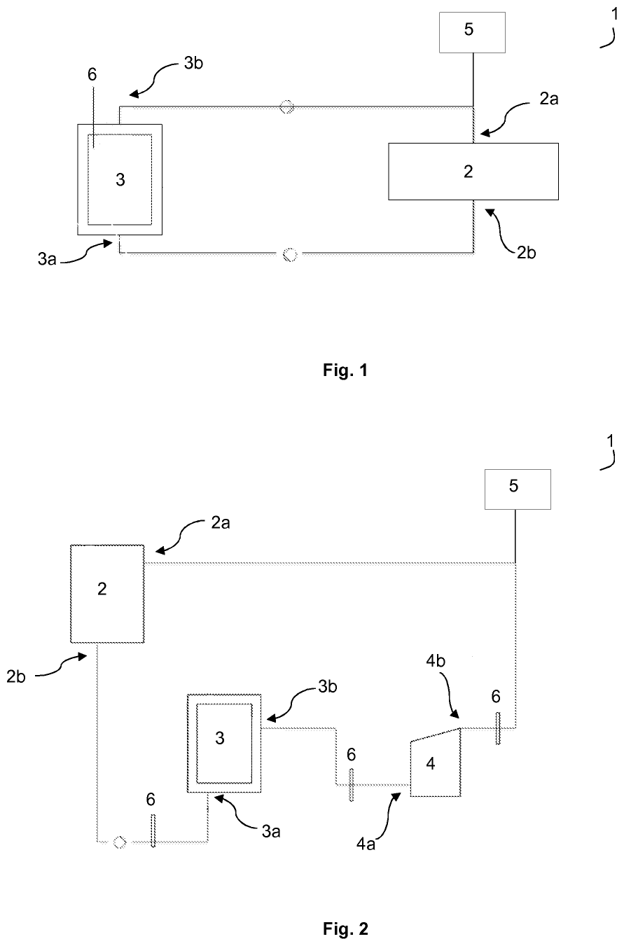

The device will be described with reference to FIGS. 1 and 2. The device 1 comprises a bioreactor wherein the culture is initially placed and then cultured.

The bioreactor 2 is preferably an aerobic bioreactor. Therefore, the bioreactor 2 is a closed or open vessel. According to some embodiments the content of the bioreactor 2 can be illuminated by an artificial light source. According to some embodiments, notably when the vessel is closed, the vessel is thus preferably transparent.

The bioreactor 2 may optionally comprise a temperature control system, including for example a cooling and/or heating device, to maintain the temperature of the culture in a predetermined temperature range such as 0-40° C., preferably 20-30° C. Otherwise, the bioreactor 2 may be operated at ambient temperature. The bioreactor 2 may also comprise a pH control system and a substrate addition system for adding substrates, e.g. nitrogen and phosphorous compounds (such as ammonium, nitrates and/or phosphates) and organic carbon, into the culture.

Furthermore, the bioreactor 2 may comprise a stirring system which may facilitate the homogenization of the culture. For example, the stirring system may comprise a system of bubbling air in the sample, or a paddle-wheel, or pumps, propellers, magnetic stirring systems.

The bioreactor 2 may have a volume from 0.003 to 20 m³, and preferably from 1 to 10 m³.

The bioreactor 2 may have an inlet 2a and an outlet 2b for the entry and the exit of a sample of the culture.

According to some embodiments, only one bioreactor 2 is present in the device 1.

According to other embodiments (not illustrated in the figures), several bioreactors 2, for example placed in parallel, are present in the device 1, the bioreactors are feeding the chamber 3 one after the other in such a case. The device 1 according to the invention also comprises at least one chamber 3 having an inlet 3a and an outlet 3b. The chamber 3 is fluidically connected to the bioreactor 2 so that the bioreactor 2 can directly supply the chamber 3 with a sample of the culture. The (direct) fluidic connection between the bioreactor 2 and the chamber 3 may be achieved with conduits, channels or tubing that link the bioreactor 2 to the chamber 3 (as illustrated in the figures).

Therefore, during its transfer from the bioreactor 2 to the chamber 3 for example by using a peristaltic pump, the sample does not come into contact with the external environment and is not transferred to an external element such as a syringe, pipette or the like.

The inlet 3a of the chamber 3 is fluidically connected to the outlet 2b of the bioreactor 2. According to some embodiments (and as illustrated in FIG. 1), the outlet 3b of the chamber 3 is fluidically connected to the inlet 2a of the bioreactor 2 in order to form a loop comprising the chamber 3 and the bioreactor 2. Alternatively, the outlet 3b of the chamber 3 may be connected to an additional chamber as described below.

The chamber 3 is sealed in order to obstruct contact of the sample therein with the external environment (to avoid oxygenation of the culture). The chamber 3 is a closed vessel. The content of the chamber 3 may be illuminated (by artificial or natural light). The walls of the chamber 3 may be partly transparent, fully transparent or opaque. Furthermore, the chamber may comprise a stirring system which may facilitate the homogenization of the culture.

The chamber 3 may have an internal volume from 0.1 to 1 L, preferably from 0.25 to 0.5 L.

The device 1 according to the invention, and more particularly the chamber 3, comprises a source for emitting light, or source of light (not illustrated in the figures) in the chamber 3. The source of light makes it possible for the microalgae to carry out photosynthesis.

The source of light may be for example a light-emitting diode lamp located inside the chamber 3. Alternatively, the source of light may be located outside of the chamber 3 (the chamber 3 being at least partly transparent). In such a case, the chamber 3 is preferably enclosed in an opaque casing to protect it from external light.

Alternatively, the chamber 3 may be at least partly transparent and the source of light may be natural external light. In this case, the chamber 3 is preferably provided with a movable opaque protective device (such as a casing or cover) configured to block the natural external light.

Preferably, the device 1 comprises only one chamber 3 with a source for emitting light. However, if the device 1 comprises more than one chamber 3 with a source of light (not illustrated in figures), the chambers 3 may be fluidically connected in series to one another. In this case, the inlet 3a of the first chamber 3 in series is fluidically connected to the outlet 2b of the bioreactor 2 while the outlet 3b of the last chamber 3 in series is optionally fluidically connected to the inlet 2a of the bioreactor 2. Alternatively, the chambers 3 may be placed in parallel. In this case, the inlet 3a of each chamber 3 is fluidically connected to the outlet 2b of the bioreactor 2, while the outlet 3b of each chamber 3 is optionally fluidically connected to the inlet 2a of the bioreactor 2.

According to some preferred embodiments, the device may further comprise at least one additional chamber 4 fluidically connected to the chamber 3 and the bioreactor 2. Therefore, the additional chamber 4 may have an inlet 4a and an outlet 4b, the inlet 4a of the additional chamber 4 being for example fluidically connected to the outlet 3b of the chamber 3 and the outlet 4b of the additional chamber 4 being fluidically connected to the inlet 2a of the bioreactor 2 in order to form a loop comprising the chamber 3, the additional chamber 4 and the bioreactor 2 (as illustrated in FIG. 2). The (direct) fluidic connection between the additional chamber 4, the chamber 3 and the bioreactor 2 may be achieved with conduits, channels or tubing.

Therefore, depending on the configuration of the device 1, the sample may circulate from the bioreactor 2 to the chamber 3 and then either back to the bioreactor 2 (as illustrated in FIG. 1) or to the additional chamber 4 and then back to the bioreactor 2 (as illustrated in FIG. 2), for example by using a peristaltic pump. Alternatively, the sample, after entering the chamber 3 and/or the additional chamber 4, may be directed to a waste vessel 5 comprised in the device 1. In this case, the device 1 may also comprise a switching valve in order to direct the sample towards the bioreactor 2 or the waste vessel 5.

Alternatively, and contrary to what is illustrated on FIG. 2, the additional chamber 4 may be placed between the bioreactor 2 and the chamber 3 in the direction of flow (instead of the chamber 3 being placed between the bioreactor 2 and the additional chamber 4). In this case, the outlet 2b of the bioreactor 2 is fluidically connected to the inlet 4a of the additional chamber 4 and the outlet 4b of the additional chamber 4 is fluidically connected to the inlet 3a of the chamber 3.

The additional chamber 4 is sealed in order to obstruct contact with the external environment. Furthermore, the additional chamber 4 does not allow light from the external environment to enter the additional chamber 4. The additional chamber 4 may comprise external walls made from a material chosen from glass or polymers. The additional chamber 4 may be enclosed in an opaque casing or covered by an opaque cover or opaque layer.

The additional chamber 4 may have an internal volume from 0.1 to 1 L, preferably from 0.25 to 0.5 L

This additional chamber 4 may preferably be devoid of a source for emitting light. Therefore, when the sample is in the additional chamber 4, not only is the sample not in contact with light from the external environment, but also no light is emitted via a source of light in the additional chamber 4, therefore, no photosynthesis can occur in chamber 4.

Preferably, the device 1 comprises only one additional chamber 4.

However, if the device 1 comprises several additional chambers 4 (not illustrated in the figures), the additional chambers 4 may be fluidically connected in series to one another. For example, when the device 1 comprises several chambers 3 (with a source of light) and several additional chambers 4, the chambers 3 and additional chambers 4 may be fluidically connected in series, each chamber 3 alternating with an additional chamber 4 (as illustrated in FIG. 2 for one chamber 3 and one additional chamber 4). In this case, the inlet 3a of the first chamber 3 in series may be fluidically connected to the outlet 2b of the bioreactor 2 while the outlet 3b of the last chamber 3 (or the outlet 4b of the last additional chamber 4) in series may be fluidically connected to the inlet 2a of the bioreactor 2.

According to other embodiments (not illustrated in the figures), several chambers 3 may be placed in parallel and a single additional chamber 4 may be placed in series; or a single chamber 3 may be placed in series relative to several additional chambers 4 placed in parallel; or several chambers 3 may be placed in parallel, with one additional chamber 4 in series with respect to each chamber 3.

Furthermore, the chamber 3 and/or the additional chamber 4 may comprise a stirring system (not illustrated in the figures), which may facilitate the homogenization of the sample.

The device 1 also comprises at least one sensor 6 for measuring the concentration of dissolved oxygen in the sample.

According to some embodiments, the device 1 comprises only one sensor 6 for measuring the concentration of dissolved oxygen.

Such sensor 6 may be an optical oxygen sensor (for example an optical oxygen sensor commercialized by the companies Hach or Hamilton).This sensor 6 may be located for example in the chamber 3 (as illustrated in FIG. 1).

Alternatively, the sensor 6 may be located upstream or downstream of the chamber 3 and more particularly proximate to the inlet 3a or the outlet 3b of the chamber 3.

According to other embodiments, the device 1 comprises several sensors 6 for measuring the concentration of dissolved oxygen. It may notably comprise two, or three, or four sensors 6 for measuring the concentration of dissolved oxygen. For example, a first sensor 6 may be located upstream (and proximate to) the inlet 3a of the chamber 3 while a second sensor 6 may be located downstream of (and proximate to) the outlet 3b of the chamber 3. When the device 1 comprises an additional chamber 4, a first sensor 6 may be located in the chamber 3 and a second sensor 6 may be located in the additional chamber 4 (not illustrated in the figures). Alternatively and preferably (as illustrated in FIG. 2), a first sensor 6 may be located upstream of (and proximate to) the inlet 3a of the chamber 3, a second sensor 6 may be located between the outlet 3b of the chamber 3 and the inlet 4a of the additional chamber 4, and a third sensor 6 may be located downstream of (and proximate to) the outlet 4b of the additional chamber 4.

The device 1 may also comprise at least one sensor for measuring the concentration of dissolved carbon dioxide in the sample (not illustrated in the figures).

Such sensor may be a sensor commercialized by the company Mettler Toledo or another equivalent

According to some preferred embodiments, the device 1 comprises only one sensor for measuring the concentration of dissolved carbon dioxide. This sensor may be located for example in the chamber 3 or the additional chamber 4.

Alternatively, the sensor may be located out of the chamber 3 (or the additional chamber 4) and more particularly upstream of the inlet 3a or downstream of the outlet 3b of the chamber 3 (or upstream of the inlet 4a or downstream of the outlet 4b of the additional chamber 4).

According to other embodiments, the device 1 comprises several sensors for measuring the concentration of dissolved carbon dioxide. It may notably comprise two, or three, or four sensors for measuring the concentration of dissolved carbon dioxide. For example, a first sensor may be located upstream of (proximate to) the inlet 3a of the chamber 3 while a second sensor may be located downstream of (proximate to) the outlet 3b of the chamber 3. When the device 1 comprises an additional chamber 4, a first sensor may be located in the chamber 3 and a second sensor may be located in the additional chamber 4. Alternatively and preferably, a first sensor may be located upstream of (proximate to) the inlet 3a of the chamber 3, a second sensor may be located between the outlet 3b of the chamber 3 and the inlet 4a of the additional chamber 4 and a third sensor may be located downstream of (proximate to) the outlet 4b of the additional chamber 4.

The device 1 may also comprise one or more sensors for measuring the pH of the sample, and/or one or more sensors for measuring the temperature of the sample, and/or one or more sensors for measuring the turbidity of the sample, and/or one or more sensors for measuring the optical density (example at 680 nm) of the sample and and/or one or more sensors for measuring the total suspended solids (TSS) of the sample (not illustrated in the figures).

A sensor for measuring the pH may be a sensor commercialized by the companies Hamilton or Hach compatible with the data acquisition system. The sensor for measuring the temperature may be the same as the one used for measuring the concentration in dissolved oxygen or the pH of the sample According to some preferred embodiments, the device 1 comprises only one sensor for measuring the pH and/or only one sensor for measuring the temperature and/or only one sensor for measuring the turbidity and/or only one sensor for measuring the optical density and and/or only one sensor for measuring the total suspended solids (TSS) of the sample. This or these sensors may preferably be located for example in the chamber 3 (or the additional chamber 4).

Alternatively, the sensor(s) may be located out of the chamber 3 (or the additional chamber 4) and more particularly upstream of the inlet 3a or downstream of the outlet 3b of the chamber 3 (or upstream of the inlet 4a or downstream of the outlet 4b of the additional chamber 4).

According to other embodiments, the device 1 comprises several sensors for measuring the pH, and/or several sensors for measuring the temperature and/or several sensors for measuring the turbidity and/or several sensors for measuring the optical density and and/or several sensors for measuring the total suspended solids (TSS) of the sample. It may notably comprise two, or three, or four sensors for measuring the pH and/or two, or three, or four sensors for measuring the temperature and/or two, or three, or four sensors for measuring the turbidity and/or two, or three, or four sensors for measuring the optical density and and/or two, or three, or four sensors for measuring the total suspended solids (TSS) of the sample. For example, a first sensor (pH sensor and/or temperature sensor and/or turbidity sensor and/or optical density sensor and/or total suspended solids sensor) may be located upstream of (proximate to) the inlet 3a of the chamber 3 while a second sensor (pH sensor and/or temperature sensor and/or turbidity sensor and/or optical density sensor and/or total suspended solids sensor) may be located downstream of (proximate to) the outlet 3b of the chamber 3. When the device 1 comprises an additional chamber 4, a first sensor (pH sensor and/or temperature sensor and/or turbidity sensor and/or optical density sensor and/or total suspended solids sensor) may be located in the chamber 3 and a second sensor (pH sensor and/or temperature sensor and/or turbidity sensor and/or optical density sensor and/or total suspended solids sensor) may be located in the additional chamber 4. Alternatively, and preferably a first sensor (pH sensor and/or temperature sensor and/or turbidity sensor and/or optical density sensor and/or total suspended solids sensor) may be located upstream of (proximate to) the inlet 3a of the chamber 3, a second sensor (pH sensor and/or temperature sensor and/or turbidity sensor and/or optical density sensor and/or total suspended solids sensor) may be located between the outlet 3b of the chamber 3 and the inlet 4a of the additional chamber 4 and a third sensor (pH sensor and/or temperature sensor and/or turbidity sensor and/or optical density sensor and/or total suspended solids sensor) may be located downstream of (proximate to) the outlet 4b of the additional chamber 4.

In addition, the device 1 may comprise at least one pump for the circulation of the sample (for example a peristaltic pump), e.g. from the bioreactor 2 to the chamber 3 and back and/or to the bioreactor 2 to the chamber 3 and then to the additional chamber 4 and back to the bioreactor 2.

According to some embodiments, the device 1 may also comprise one or more injectors. These injectors may be used for example to introduce components such as substrates and/or inhibitors (or toxic substances) into the fluid circuitry, e.g. into one of the chambers 3 and/or additional chambers 4. According to some embodiments, the injectors may be configured to perform an injection directly into the chamber(s) 3 and/or the additional chamber(s) 4.

According to other embodiments, the injectors may be configured to perform an injection into conduits, channels or tubing connecting the chamber(s) 3 and/or the additional chamber(s) 4 with the bioreactor 2 and/or leading to the chamber(s) 3 and/or the additional chamber(s) 4.

Each injector may be fluidically connected to one or more tanks containing components such as substrates and/or inhibitors (or toxic substances).

The device 1 may comprise a number of opening/closing valves to initiate of stop flow from one part of the device 1 to the other.

According to some embodiments, the chamber 3, the source for emitting light in the chamber and at least one oxygen sensor can form an apparatus that is configured to be connected, via the chamber 3 to the bioreactor 2 to form the device 1. The apparatus may have the same features as the device 1, as described above. The apparatus may be portable.

### Method for Characterizing the Activity of a Culture Comprising Microalgae

The invention also relates to a method for characterizing the activity (notably by measuring the concentration of dissolved oxygen or carbon dioxide) of a culture comprising microalgae and optionally bacteria, the culture being as described above. This method is preferably implemented in the device 1 described above.

The method may comprise a step of initially placing the culture in a bioreactor 2. According to some embodiments, prior to placing the culture in the bioreactor 2, the method may comprise a step of inoculation and a step of growth of the culture (microalgae and/or bacteria). According to some embodiments, the method may comprise a step of injecting substrates for the microalgae and optionally for the bacteria into the bioreactor 2 in order to enhance the culture. The culture may remain and be cultivated in the bioreactor 2 for a period of time. This period of time may be for example from several days (for example 3 or 4 or 5 or 6 or 7 or more than 7 days) to several years (for example 1 or 2 or 3 or 4 or 5 or more than 5 years)

Then, a sample of the culture (or an amount of the culture) is directly supplied by using a circulation (peristaltic) pump from the bioreactor 2 to a chamber 3. The supply may be carried out via the conduits, channels or tubing that link the bioreactor 2 to the chamber 3.

The sample in the chamber 3 may have a volume from 0.1 to 1, preferably from 0.25 to 0.5 L.

According to some embodiments, the supply of the chamber 3 with the sample may be carried out in a discontinuous manner. In other words, an amount of culture may be supplied to the chamber 3. Then flow from the bioreactor 2 to the chamber may be stopped. Then the sample may be discharged (e.g. to waste, or to an additional chamber 4) or returned to the bioreactor 2 (optionally while a new sample may be supplied from the bioreactor 2 to the chamber 3). The device of the embodiment of FIG. 1 is particularly adapted for such a discontinuous mode of operation.

According to other embodiments, the supply of the chamber 3 with the sample may be carried out in a continuous manner. In other words, an amount of culture may be continuously supplied to the chamber 3 while at the same time an amount of culture may be continuously discharged (e.g. to waste, or to an additional chamber 4) or returned to the bioreactor 2. The device of the embodiment of FIG. 2 is particularly adapted for such a continuous mode of operation.

The method further comprises a step of illuminating the sample in the chamber 3. The illumination may be applied for a period of time varying from 1 minute to several hours (for example 1 or 2 or 3 or 4 or 5 or more than 5 hours).

According to some embodiments, the method according to the invention comprises one step of illuminating the sample (in the chamber 3).

According to other, preferred embodiments, the method according to the invention comprises several steps of illuminating the sample, for example two, or three, or four or five, or ten, or fifteen, or twenty steps of illuminating the sample. For example, a sample can be supplied to the chamber 3 in order to carry out a first step of illuminating the sample (dissolved oxygen concentration in the illuminated sample being measured). Then the sample can be discharged or returned to the bioreactor 2 and a new sample can be supplied to the chamber 3 from the bioreactor 2 in order to carry out a second step of illuminating the (new) sample (dissolved oxygen concentration in the illuminated sample being measured). In this case, the steps of illuminating the sample are successive and the sample is renewed before each step of illuminating the sample.

Alternatively, other steps (as described below) may be present between two steps of illuminating the sample, in other words the two steps of illuminating the sample are not successive.

During the step of illuminating the sample, the microalgae carry out photosynthesis to produce oxygen. At the same time, they consume oxygen for their respiration (negligible compared to produced oxygen).

According to some embodiments, during the illumination of the sample, at least one substrate is present in the sample (e.g. is added to the sample). Such substrate may be a substrate for microalgae. Alternatively and preferably, and when bacteria are present in the culture, such substrate may be a substrate for bacteria. Some substrates can be substrates both for microalgae and bacteria. The addition of the substrate(s) for example by injecting the substrate(s) into the chamber 3 or into a conduit/channel/tubing leading to the chamber 3, makes it possible to maximize the activity of microalgae and/or bacteria in order to measure the concentration of dissolved oxygen under these (maximized) conditions. Such substrate may be for example ammonium chloride, sodium nitrite, ethanol, sodium acetate, glycerol.

Such substrate(s) may have a concentration in the sample varying from 1 to 400 ppm and preferably from 3 to 200 ppm.

According to some embodiments, during the illumination of the sample, at least one inhibitor or toxic compound is present in the sample (e.g. is added to the sample). Such inhibitor or toxic compound may be an inhibitor or toxic compound for microalgae. Alternatively and preferably, such inhibitor or toxic compound may be an inhibitor or toxic compound for bacteria, when bacteria are present in the culture. The addition of the inhibitor(s) or toxic compound(s) for example by injecting the inhibitor(s) or toxic compound(s) into the chamber 3 or into a conduit/channel/tubing leading to the chamber 3, makes it possible to deactivate the bacteria present in the culture in order to measure the concentration of dissolved oxygen which corresponds solely to the activity of microalgae (the production of oxygen due to photosynthesis and consumption of oxygen due to microalgae respiration). For example, the addition of inhibitor(s) or toxic compound(s) may deactivate all bacteria present in the sample. Otherwise, notably when heterotrophic and autotrophic bacteria are present in the sample, the addition of specific inhibitor(s) or toxic compound(s) may deactivate only autotrophic bacteria while heterotrophic bacteria remain activated. Alternatively, the addition of specific inhibitor(s) or toxic compound(s) may deactivate only heterotrophic bacteria while autotrophic bacteria remain activated. Such inhibitors or toxic compound(s) may be for example copper sulfate, potassium chlorate, sodium chlorate, allylthiourea, naphthalene, toluene, methanol, glutaraldehyde, a quaternary ammonium salt, THPS (tetrakis(hydroxymethyl)phosphonium sulfate), ethylene glycol, 2-butoxyethanol, 4-tert-octylphenol.

According to some embodiments, during the illumination of the sample, only substrate(s) are present in (e.g. added to) the sample.

According to some embodiments, during the illumination of the sample, only inhibitor(s) or toxic compound(s) are present in (e.g. added to) the sample.

According to other embodiments, during the illumination of the sample, substrate(s) and inhibitor(s) or toxic compound(s) are present in (e.g. added to) the sample.

For example, during a first step of illuminating the sample, substrate(s) may be present in (e.g. added to) the sample and during a second step of illuminating the sample, inhibitor(s) or toxic compound(s) may be present in (e.g. added to) the sample.

Alternatively, during a first step of illuminating the sample, substrate(s) may be present in (e.g. added to) the sample and during a second step of illuminating the sample, inhibitor(s) or toxic compound(s) and substrate(s) may be present in (e.g. added to) the sample.

The concentration of dissolved oxygen in the sample is measured.

Based on the measurement of dissolved oxygen, a rate of oxygen production by the illuminated sample and/or a rate of oxygen consumption by the non-illuminated sample may be determined. The measurements may take place during the one or more steps of illuminating the sample and/or the one or more steps during which the sample is kept in the absence of light, or before and after these steps.

The “rate of oxygen production” has a negative value if net oxygen is overall consumed in the sample, and has a positive value if net oxygen is overall produced in the sample. It may be expressed for instance in mg O₂/(L·h).

The “rate of oxygen consumption” has a positive value if net oxygen is overall consumed in the sample, and has a negative value if net oxygen is overall produced in the sample. It may be expressed for instance in mg O₂/(L·h). In other terms, the rate of oxygen consumption corresponds to a negative rate of oxygen production.

The rate of oxygen production or consumption may for example be determined by calculating the derivative of the concentration of dissolved oxygen as a function of time and optionally by averaging said derivative over a period of time; or by calculating the difference in the concentration of dissolved oxygen between two points in time and dividing it by the interval between said points in time.

Making reference to FIG. 1, this measurement may be carried out, when the sample is supplied in a discontinuous way (as explained above) in the chamber 3, for example with a sensor located in the chamber 3. For example, when the step of illuminating the sample is carried out for a certain duration, the rate of oxygen production of the illuminated sample may be determined by calculating the derivative of the concentration of dissolved oxygen measured during the step of illuminating the sample as a function of such duration.

Alternatively, making reference to FIG. 2, this measurement may be carried out, when the sample is supplied in a continuous way (as explained above), before the sample is supplied to the chamber 3 and after it is discharged from the chamber 3 (therefore before and after the illumination step), for example with a sensor 6 located upstream of (proximate to) the inlet 3a and a sensor 6 located downstream of (proximate to) the outlet 3b of said chamber 3. The difference between the measurements obtained from both sensors makes it possible to determine the rate of oxygen production by the illuminated sample, taking account the flow rate in the system and the volume of the chamber 3.

After such measurement(s), the sample may be directed to a waste vessel 5. Alternatively, the sample may return to the bioreactor 2. Alternatively and preferably, the sample may be subjected to at least one step of keeping the sample in the absence of light for example in the additional chamber 4. During said step, the sample is kept in the dark, in other words, light is not provided to the sample.

Making for example reference to FIG. 1, the step of keeping the sample in the absence of light may be carried out in the same chamber 3 where the illumination step(s) are carried out. In this case, the light source stops illuminating the sample during the step of keeping the sample in the absence of light. This is preferable when the sample is provided to the chamber 3 in a discontinuous way.

Alternatively, and making for example reference to FIG. 2, the step of keeping the sample in the absence of light may be carried out in an additional chamber 4, different from the chamber 3 where the illumination step(s) are carried out. This is preferable when the sample is provided to the chamber 3 in a continuous way. In this case, the sample may be transferred from the chamber 3 to the additional chamber 4 via a channel, or a conduit, or tubing fluidically connecting the chamber 3 and the additional chamber 4.

According to some embodiments, the method according to the invention comprises one step of keeping the sample in the absence of light.

According to other, preferred embodiments, the method according to the invention comprises several steps of keeping the sample in the absence of light, for example two, or three, or four or five, or ten, or fifteen, or twenty steps of keeping the sample in the absence of light.

For example, the sample located in the chamber 3 (for example after undergoing one or more illumination steps) can undergo a first step of keeping the sample in the absence of light (during which the measurement of the dissolved oxygen concentration is carried out). Then the sample can be discharged or returned to the bioreactor 2 and a new sample can be supplied to the chamber 3 from the bioreactor 2 in order to carry out a second step of keeping the (new) sample in the absence of light (during which measurement of the dissolved oxygen concentration is carried out). In this case, the steps of keeping the sample in the absence of light are successive and the sample is renewed before each step of keeping the sample in the absence of light.

Alternatively, a first step of keeping the sample in the absence of light may be carried out in chamber 3, and then the sample may be transferred to another chamber (for example the additional chamber 4) to carry out a second step of keeping the sample in the absence of light. In this case, the two steps of keeping the sample in the absence of light are successive, but the sample is not renewed before its transfer to the other chamber.

During the step of keeping the sample in the absence of light, microalgae are not able to carry out photosynthesis. The concentration of dissolved oxygen in the sample corresponds to the consumption of oxygen related to the respiration of microalgae as well as to the consumption of oxygen related to the respiration of bacteria when they are present in the sample.

According to some embodiments, during the step of keeping the sample in the absence of light, at least one substrate can be present in (e.g. added to) the sample. Such substrate may be as described above. The addition of the substrate (s) for example by injecting the substrate (s) into the chamber 3 (or the additional chamber 4) or into a conduit/channel/tubing leading to the chamber 3 (or the additional chamber 4), makes it possible to maximize the activity of microalgae and/or bacteria in order to measure the concentration of dissolved oxygen under these (maximized) conditions.

According to some embodiments, during the step of keeping the sample in the absence of light, at least one inhibitor or toxic compound can be present in (e.g. added to) the sample. Such inhibitor or toxic compound may be as described above. The addition of the inhibitor(s) or toxic compound(s) for example by injecting the inhibitor(s) or toxic compound(s) into the chamber 3 (or the additional chamber 4) or into a conduit/channel/tubing leading to the chamber 3 (or the additional chamber 4), makes it possible to deactivate the bacteria present in the culture in order to measure the concentration of dissolved oxygen which corresponds solely to the oxygen consumed due to the respiration of microalgae.

For example, the addition of inhibitor(s) or toxic compound(s) may deactivate all bacteria present in the sample. Otherwise, notably when heterotrophic and autotrophic bacteria are present in the sample, the addition of specific inhibitor(s) or toxic compound(s) may deactivate only autotrophic bacteria while heterotrophic bacteria remain activated. Alternatively, the addition of specific inhibitor(s) or toxic compound(s) may deactivate only heterotrophic bacteria while autotrophic bacteria remain activated.

According to some embodiments, during the step of keeping the sample in the absence of light, only substrate(s) are present in (e.g. added to) the sample.

According to some embodiments, during the step of keeping the sample in the absence of light, only inhibitor(s) or toxic compound(s) are present in (e.g. added to) the sample.

According to other embodiments, during the step of keeping the sample in the absence of light, substrate(s) and inhibitor(s) or toxic compound(s) are present in (e.g. added to) the sample.

For example, during a first step of keeping the sample in the absence of light, substrate(s) may be present in (e.g. added to) the sample and during a second step of keeping the sample in the absence of light, inhibitor(s) or toxic compound(s) may be present in (e.g. added to) the sample.

Alternatively, during a first step of keeping the sample in the absence of light, substrate(s) may be present in (e.g. added to) the sample and during a second step of keeping the sample in the absence of light, inhibitor(s) or toxic compound(s) and substrate(s) may be present in (e.g. added to) the sample.

The concentration of dissolved oxygen in the sample may be measured so as to determine, a rate of oxygen consumption by the sample in the absence of light. The measurements may take place during the one or more steps of keeping the sample in the absence of light, or before and after these steps. Making reference to FIG. 1, this measurement may be carried out, when the sample is supplied in a discontinuous way (as explained above) in the chamber 3, for example with a sensor 6 located in the chamber 3. For example, when the step of keeping the sample in the absence of light is carried out for a certain duration, the rate of oxygen consumption of the non-illuminated sample may be determined by calculating the derivative of the concentration of dissolved oxygen measured during the step of keeping the sample in the absence of light as a function of such duration.

Alternatively, making reference to FIG. 2, this measurement may be carried out, when the sample is supplied in a continuous way (as explained above), before the sample is supplied to the additional chamber 4 and after it is discharged from the additional chamber 4 (therefore before and after the step of keeping the sample in the absence of light), for example with a sensor 6 located upstream of (proximate to) the inlet 4a and a sensor 6 located downstream of (proximate to) the outlet 4b of said additional chamber 4. The difference between the measurements obtained from both sensors makes it possible to determine the rate of oxygen consumption by the sample in the absence of light, taking account the flow rate in the system and the volume of the additional chamber 4. According to some embodiments, one step of keeping the sample in the absence of light can be carried out after one step of illuminating the sample. According to alternative embodiments, one step of keeping the sample in the absence of light can be carried out prior to one step of illuminating the sample. When several steps of illuminating the sample are carried out and several steps of keeping the sample in the absence of light are carried out, it is preferable that each illumination step alternates with a step of keeping the sample in the absence of light. The alternation may be achieved by switching the source of light on and off, or by passing the sample from a chamber where it is illuminated to another chamber or portion of the device where it is not illuminated. In case the alternation is achieved by switching the source of light on and off, after the illumination step (and the measurement of dissolved oxygen concentration), the sample may be discharged or returned to the bioreactor 2 and be replaced by a new sample which may undergo the step of keeping the (new) sample in the absence of light (and measure its concentration of dissolved oxygen). In case the alternation is achieved by passing the sample from a chamber where it is illuminated to another chamber or portion of the device where it is not illuminated, the same sample may be used for both steps and both measurements.

Furthermore, when several steps of illuminating the sample are carried out and when several steps of keeping the sample in the absence of light are carried out and when each illumination step alternates with at least one step of keeping the sample in the absence of light, for each alternation the sample can remain the same (i.e. the bioreactor 2 does not supply a new sample between a succession of an illumination step and a step of keeping the sample in the absence of light).

Alternatively, when several steps of illuminating the sample are carried out and when several steps of keeping the sample in the absence of light are carried out and when each illumination step alternates with at least one step of keeping the sample in the absence of light, the bioreactor 2 can supply a new sample for each alternation (in other words for each couple of an illumination step and a step of keeping the sample in the absence of light).

The method according to the invention may also comprise a step for discharging the sample into a waste vessel 5 via a conduit or a channel or tubing. Alternatively, the method may comprise a step of returning the sample into the bioreactor 2 via a conduit or a channel or tubing.

According to some preferred embodiments and with reference to FIG. 1, the method according to the invention comprises the supply from the bioreactor 2 of a sample comprising microalgae and bacteria to the chamber 3, in a discontinuous manner. This method further comprises the application of a first illumination step, during which at least one substrate (preferably a bacteria substrate) is preferably present in or added to the sample, and the measurement of the concentration of dissolved oxygen corresponding to the production of oxygen due to the microalgae photosynthesis and the consumption of oxygen due to the respiration of microalgae and bacteria. Dissolved oxygen may be measured with a sensor 6 located in the chamber 3. Then the sample can be discharged (for example in a waste vessel 5) or returned to the bioreactor 2 and a new sample can be supplied to chamber 3. These steps can optionally be repeated several times.

Then, a first step of keeping the sample in the absence of light can be carried out (i.e. the source of light is switched off), preferably with at least one substrate (preferably a bacteria substrate) present in or added to the sample, and the measurement of the concentration of dissolved oxygen is performed, which this time corresponds to the consumption of oxygen due to the respiration of microalgae and bacteria. Dissolved oxygen may be measured with the same sensor 6 located in the chamber 3. At the end of this step, the sample can be discharged (for example in a waste vessel 5) or returned to the bioreactor 2 and a new sample can be supplied to chamber 3. These steps can optionally be repeated several times.

Optionally, a second illumination step may then be carried out in the presence of at least one inhibitor or toxic compound (preferably a bacteria inhibitor or toxic compound) and optionally at least one substrate (preferably a bacteria substrate when for example the inhibitor or toxic compound deactivates only one bacteria type) in the sample, and the concentration of dissolved oxygen (corresponding to the production of oxygen due to the microalgae photosynthesis and the consumption of oxygen due to the respiration of microalgae and optionally the respiration of bacteria that have not been inhibited) is measured. For instance, the at least one inhibitor or toxic compound (and optionally the at least one substrate) may be added between the first step of keeping the sample in the absence of light and the second illumination step or at the onset of the second illumination step or throughout the second illumination step. Dissolved oxygen may be measured with the same sensor 6 located in the chamber 3 and the sample can then be discharged (for example in a waste vessel 5) or returned to the bioreactor 2 and a new sample can be supplied to chamber 3. These steps can optionally be repeated several times.

Optionally, a second step of keeping the sample in the absence of light can then be carried out in the presence of at least one inhibitor or toxic compound (preferably a bacteria inhibitor or toxic compound) and optionally at least one substrate (preferably a bacteria substrate when for example the inhibitor or toxic compound deactivates only one bacteria type) in the sample, and the concentration of dissolved oxygen (this time corresponding to the consumption of oxygen due to the respiration of microalgae and optionally the respiration of bacteria that have not been inhibited) is measured. Optionally, the at least one inhibitor or toxic compound and/or the at least one substrate may be added between the second illumination step and the second step of keeping the sample in the absence of light or at the onset of the second step of keeping the sample in the absence of light or throughout the second step of keeping the sample in the absence of light. Dissolved oxygen may be measured with the same sensor 6 located in the chamber 3. At the end of these steps, the sample can exit the chamber 3 in order to be discharged to a waste vessel 5 or in order to return to the bioreactor 2. These steps can optionally be repeated several times.

Therefore, according to these embodiments, the method according to the invention is carried out in a discontinuous manner, in other words, a sampled amount of culture circulates from the bioreactor 2 to the chamber 3 once in order to carry out the necessary measurements. After the measurements have been completed, the sample is transferred back to the bioreactor 2 (or the waste vessel 5).

According to these embodiments, adding the rate of oxygen production in a step of illuminating the sample (rate of oxygen production of the illuminated sample) and the rate of oxygen consumption in a step of keeping the sample in the absence of light (rate of oxygen consumption of the non-illuminated sample) makes it possible to determine the rate of oxygen production by microalgal photosynthesis (as the microalgal and bacterial respiratory contribution eliminated in this operation). For example, with reference to the previous paragraphs, the rate of oxygen production by microalgal photosynthesis can be determined by calculating the difference between the rate of oxygen production in the first step(s) of illuminating the sample and the rate of oxygen consumption in the first step(s) of keeping the sample in the absence of light, or by calculating the difference between the rate of oxygen production in the second step(s) of illuminating the sample and the rate of oxygen consumption in the second step(s) of keeping the sample in the absence of light.

Still, according to these embodiments, the difference between the rate of oxygen consumption in the first step(s) of keeping the sample in the absence of light and the rate of oxygen consumption in the second step(s) of keeping the sample in the absence of light or the difference between the rate of oxygen production in the first illumination step(s) and the rate of oxygen production in the second illumination step(s) makes it possible to calculate the rate of oxygen consumption by the bacteria subject to inhibition by the inhibitor(s) or toxic compound(s).

According to other preferred embodiments and with reference to FIG. 2, the method according to the invention comprises the supply from the bioreactor 2 of a sample comprising microalgae and bacteria to the chamber 3, in a continuous manner.

This method further comprises the application of a first illumination step during which at least one substrate (preferably a bacteria substrate) is optionally added to the sample, prior to its entrance into the chamber 3 (for instance into a channel leading to the chamber 3) and the concentration of dissolved oxygen is measured with a first sensor 6 located upstream of (proximate to) the inlet 3a and a second sensor 6 located downstream of (proximate to) the outlet 3b of the chamber 3. Taking into account the volume of the chamber 3 and the flow rate in the device, the difference between the two measurements makes it possible to determine the rate of oxygen production of the sample in the chamber 3. This rate of oxygen production takes into account oxygen production due to microalgal photosynthesis and oxygen consumption due to microalgal and optionally bacterial respiration.

Then a first step of keeping the sample in the absence of light can be carried out during which at least one substrate (preferably a bacteria substrate) is optionally added to the sample prior to its entrance into the additional chamber 4 (for example into a channel leading to the additional chamber 4—although the addition of substrate performed for the first illumination step may be sufficient). This time, the concentration of dissolved oxygen is measured with the second sensor 6 and a third sensor 6 located downstream of (proximate to) the outlet 4b of the additional chamber 4. Taking into account the volume of the additional chamber 4 and the flow rate in the device, the difference between the two measurements makes it possible to determine the rate of oxygen consumption of the sample in the chamber 3. This rate of oxygen consumption takes into account oxygen consumption due to microalgal and optionally bacterial respiration (in the absence of microalgal photosynthesis).

The sample may be continuously transferred back to the bioreactor 2.

A second illumination step and second step of keeping the sample in the absence of light may subsequently be carried out (after performing the first illumination step and first step of keeping the sample in the absence of light), during which at least one inhibitor or toxic compound (preferably a bacteria inhibitor or toxic compound) and optionally at least one substrate (preferably a bacteria substrate when for example the inhibitor or toxic compound deactivates only one bacteria type) are added to the sample (for example into a channel leading to the chamber 3). The concentration of dissolved oxygen is measured with the first and the second sensors 6 as described above. Taking into account the volume of the chamber 3 and the flow rate in the device, the difference between the two measurements makes it possible to determine the rate of oxygen production of the sample in the chamber 3. This rate of oxygen production takes into account oxygen production due to microalgal photosynthesis and oxygen consumption due to microalgal respiration (and optionally bacterial respiration if part of the bacteria in the sample are not inhibited).

The sample is then directed towards the additional chamber 4, where a second step of keeping the sample in the absence of light can be carried out, during which at least one inhibitor or toxic compound (preferably a bacteria inhibitor or toxic compound) and optionally at least one substrate (preferably a bacteria substrate when for example the inhibitor or toxic compound deactivates only one bacteria type) are added to the sample (for example into a channel leading to the additional chamber 4—although the addition of inhibitor or toxic compound/substrate performed for the second illumination step may be sufficient). The concentration of dissolved oxygen is measured with the second and the third sensors 6 as described above. Taking into account the volume of the additional chamber 4 and the flow rate in the device, the difference between the two measurements makes it possible to determine the rate of oxygen consumption of the sample in the additional chamber 4. This rate of oxygen consumption takes into account oxygen consumption due to microalgal respiration (and optionally bacterial respiration if part of the bacteria in the sample are not inhibited).

After exiting the additional chamber 4, the sample can be discharged to a waste vessel 5 or return to the bioreactor 2.

According to these embodiments, adding the rate of oxygen production in a step of illuminating the sample (rate of oxygen production of the illuminated sample) and the rate of oxygen consumption in a step of keeping the sample in the absence of light (rate of oxygen consumption of the non-illuminated sample) makes it possible to determine the rate of oxygen production by microalgal photosynthesis (as the microalgal and bacterial respiratory contribution is eliminated in this operation). For example, with reference to the previous paragraphs, the rate of oxygen production by microalgal photosynthesis can be determined by adding the rate of oxygen production in the first step of illuminating the sample and the rate of oxygen consumption in the first step of keeping the sample in the absence of light, or by adding the rate of oxygen production in the second step of illuminating the sample and the rate of oxygen consumption in the second step of keeping the sample in the absence of light.

Still, according to these embodiments, the difference between the rate of oxygen consumption in the first step of keeping the sample in the absence of light and the rate of oxygen consumption in the second step of keeping the sample in the absence of light or the difference between the rate of oxygen production in the first illumination step and the rate of oxygen production in the second illumination step makes it possible to determine the rate of oxygen consumption by the bacteria subject to inhibition by the inhibitor(s) or toxic compound(s).

During at least one of the above steps of illuminating the sample and/or the above steps of keeping the sample in the absence of light, the method may further comprise a step of performing a measurement of the concentration of dissolved carbon dioxide in the sample. The concentration of dissolved carbon dioxide may for instance be measured similarly to the concentration of dissolved oxygen as described above.

Based on the measurement of dissolved carbon dioxide, a rate of carbon dioxide consumption may be determined, during the one or more illumination steps, similarly to what is described above in relation to the rate of oxygen production and/or a rate of carbon dioxide production may be determined, during the one or more steps during which the sample is kept in the absence of light, similarly to what is described above in relation to the rate of oxygen consumption or production.

Based on additions or subtractions of the rate of carbon dioxide production or consumption in different steps, the rate of carbon dioxide consumption due to microalgal photosynthesis may be determined, and/or the rate of carbon dioxide production due to bacterial respiration (related to bacteria which are inhibited by the inhibitor or toxic compound) may be determined, similarly to what is described above in relation to the oxygen consumption or production.

According to some embodiments the method may further comprise a step of carrying out at least one measurement of the pH of the sample.

According to some embodiments the method may further comprise a step of carrying out at least one measurement of the turbidity of the sample.

According to some embodiments the method may further comprise a step of carrying out at least one measurement of the optical density of the sample.

According to some embodiments the method may further comprise a step of carrying out at least one measurement of content in total suspended solids in the sample.

According to some embodiments, the pH of the sample in the chamber 3 and/or the additional chamber 4 may be from 5 to 9. When the pH of the sample gets lower than a minimum value of e.g. 5, a base such as sodium hydroxide may be added to the sample in order to increase the pH of the sample. When the pH of the sample gets higher than a maximum value of e.g. 9, an acid such as carbon dioxide or a solution of sulfuric acid may be added to the sample in order to decrease the pH of the sample.

### Computer-Assisted Implementation

The method may be computer-implemented. This means that steps (or substantially all the steps) of the method may be executed by at least one computer, or any like system. Thus, steps of the method may be performed by the computer, possibly fully automatically, or semi-automatically. In some variations, the triggering of at least some of the steps of the method may be performed through user-computer interaction. The level of user-computer interaction required may depend on the level of automatism foreseen and put in balance with the need to implement user's wishes. In examples, this level may be user-defined and/or pre-defined.

A typical example of computer-implementation of a method is to perform the method with a system adapted for this purpose. The system may comprise a processor coupled to a memory and a graphical user interface (GUI), the memory having recorded thereon a computer program comprising instructions for performing the method. The memory may also store a database. The memory is any hardware adapted for such storage, possibly comprising several physical distinct parts (e.g. one for the program, and possibly one for the database).

In some embodiments, the system is a client computer system, e.g. a workstation of a user.

The client computer system may receive inputs from the various sensors described above and may send output signals notably to various mechanical and/or electrical elements such as valves, pumps, injectors and light-emitting sources, in order to perform the above method. It may also store and/or display various measurement results to the user.

The client computer of the example may comprise a central processing unit (CPU) connected to an internal communication BUS, a random access memory also connected to the BUS. The client computer may further be provided with a graphical processing unit (GPU) which is associated with a video random access memory connected to the BUS. Video RAM is also known in the art as frame buffer. A mass storage device controller may manage accesses to a mass memory device, such as a hard drive. Mass memory devices suitable for tangibly embodying computer program instructions and data include all forms of nonvolatile memory, including by way of example semiconductor memory devices, such as EPROM, EEPROM, and flash memory devices; magnetic disks such as internal hard disks and removable disks; magneto-optical disks; and CD-ROM disks. Any of the foregoing may be supplemented by, or incorporated in, specially designed ASICs (application-specific integrated circuits). A network adapter may manage accesses to a network. The client computer may also include a haptic device such as cursor control device, a keyboard or the like. A cursor control device may be used in the client computer to permit the user to selectively position a cursor at any desired location on a display. In addition, the cursor control device may allow the user to select various commands, and input control signals. The cursor control device may include a number of signal generation devices for input control signals to system. Typically, a cursor control device may be a mouse, the button of the mouse being used to generate the signals. Alternatively or additionally, the client computer system may comprise a sensitive pad, and/or a sensitive screen.

The computer program may comprise instructions executable by a computer, the instructions comprising means for causing the above system to perform the method. The program may be recordable on any data storage medium, including the memory of the system. The program may for example be implemented in digital electronic circuitry, or in computer hardware, firmware, software, or in combinations of them. The program may be implemented as an apparatus, for example a product tangibly embodied in a machine-readable storage device for execution by a programmable processor. Method steps may be performed by a programmable processor executing a program of instructions to perform functions of the method by operating on input data and generating output. The processor may thus be programmable and coupled to receive data and instructions from, and to transmit data and instructions to, a data storage system, at least one input device, and at least one output device. The application program may be implemented in a high-level procedural or object-oriented programming language, or in assembly or machine language if desired. In any case, the language may be a compiled or interpreted language. The program may be a full installation program or an update program. Application of the program on the system results in any case in instructions for performing the method.

### EXAMPLE

The following example illustrates the invention without limiting it.

### Example 1

A sample of a culture comprising microalgae of the type Nannochloropsis oculata sp in a concentration of 0.3 g/L.

was transferred from a bioreactor to a chamber having a volume of 300 mL and comprising a magnetic stirrer and a source for emitting light. The source for emitting light comprised light-emitting diode strings located around the chamber.

The sample had a pH from 7.5 to 8.5 and comprised 110 ppm of nitrate ions, 7 ppm of phosphate ions. The sample had a salinity of 50 g/L.

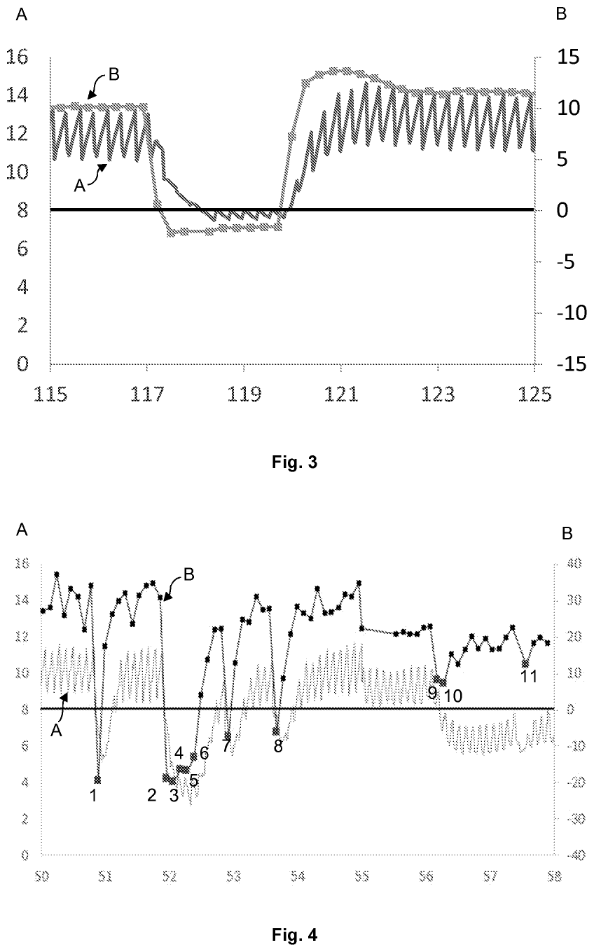

During 117 h, several illumination steps were carried out successively, in order to conduct, during each illumination step, the measurement of the dissolved oxygen present in the sample. This measurement was carried out with a Luminescent Dissolved Oxygen (LDO) sensor (HACH) inserted in the chamber. After each illumination step, the sample was returned to the bioreactor and replaced with a new sample. Therefore, as shown in FIG. 3, until 117 h, each upward slope of curve (A) corresponds to an illumination step (where the dissolved oxygen concentration increases due to photosynthesis) and each downward slope of curve (A) corresponds to the return of the sample in the bioreactor and its replacement with a new sample (which starts its photosynthesis process). From 117 to 120 h, a series of steps of keeping the sample in the absence of light was carried out during which the concentration of dissolved oxygen (A) and the oxygen consumption rate (B) are decreased due to the absence of photosynthesis and the consumption of dissolved oxygen for the respiration of microalgae. After 120 h, a series of successive illumination steps was carried out during which, the concentration of dissolved oxygen and the oxygen production rate increased. Again, each upward slope of curve (A) corresponds to an illumination step (where the dissolved oxygen concentration increases due to photosynthesis) and each downward slope of curve (A) corresponds to the return of the sample in the bioreactor and its replacement with another sample (which starts its photosynthesis process).

### Example 2

A sample of a culture comprising microalgae of the type Nannochloropsis oculata sp. in a concentration of 0.3 g/L. and aerobic bacteria in a concentration of 0.05 g/L was transferred from a bioreactor to a chamber having a volume of 300 mL and comprising a magnetic stirrer and a source for emitting light. The source for emitting light comprised light-emitting diode strings located around the chamber. The sample had a pH from 7.5 to 8.5 and comprised 110 ppm of nitrate ions, 7 ppm of phosphate ions. The sample had a salinity of 50 g/L.

During the whole experiment, the chamber was provided with light. In other words, several illumination steps were carried out successively, in order to conduct, during each illumination step, the measurement of the dissolved oxygen present in the sample. This measurement was carried out with a Luminescent Dissolved Oxygen (LDO) sensor (HACH) inserted in the chamber. After each illumination step, the sample was returned to the bioreactor and replaced with a new sample. Therefore, as shown in FIG. 4, each upward slope of curve (A) corresponds to an illumination step (where the dissolved oxygen concentration increases due to photosynthesis) and each downward slope of curve (A) corresponds to the return of the sample in the bioreactor and its replacement with a new sample.

As shown in FIG. 4, points 1 to 11 correspond to successive additions of ethanol as a substrate for the bacteria and CuSO₄ as a bacteria inhibitor. More particularly, after point 11, a total amount of 70 mg/L of CuSO₄ was added to the sample. Therefore, as shown in FIG. 4, after each addition (1 to 11) the concentration of dissolved oxygen (A) as well as the oxygen consumption rate (B) decrease due to the presence of the substrate which increases the activity of bacteria and therefore oxygen consumption due to their respiration. After the substrate is consumed, the concentration of dissolved oxygen (A) increases until the next addition. As time passes, the presence of CuSO4 which accumulates in the sample has an increasing inhibitory effect on the activity of bacteria. Therefore, despite the successive additions of the substrate, the activity of bacteria decreases from point 1 to point 11 (increase in oxygen production rate (B)).

1-39. (canceled)

40. A method for characterizing the activity of a culture comprising microalgae, the method comprising the steps of:
supplying a sample of the culture from a bioreactor (2) to a chamber (3) fluidically connected to the bioreactor (2);
illuminating the sample in the chamber (3); and
measuring the concentration of dissolved oxygen in the sample in order to determine a rate of oxygen production by the illuminated sample.

41. The method according to claim 40, further comprising the steps of:
keeping a sample of the culture from the bioreactor (2) in the absence of light; and
measuring the concentration of dissolved oxygen in said sample in order to determine a rate of oxygen consumption by the sample in the absence of light.

42. The method according to claim 41, wherein the sample kept in the absence of light is the same as the sample which is illuminated or wherein the sample kept in the absence of light is different from the sample which is illuminated.

43. The method according to claim 41, wherein the step of keeping a sample in the absence of light is carried out in the chamber (3) and/or wherein the step of keeping a sample in the absence of light is carried out in an additional chamber (4) fluidically connected to the chamber (3) and/or the bioreactor (2).

44. The method according to claim 41, wherein the rate of oxygen production by microalgal photosynthesis is determined by adding the rate of oxygen production of the illuminated sample and the rate of oxygen consumption of the sample in the absence of light.

45. The method according to claim 41, wherein the concentration of dissolved oxygen in the sample is measured during the step of illuminating the sample and/or during the step of keeping a sample in the absence of light.

46. The method according to claim 41, wherein the rate of oxygen production by the illuminated sample is determined by measuring the concentration of dissolved oxygen in the sample before and after the step of illuminating the sample, and/or wherein the rate of oxygen consumption by the sample in the absence of light is determined by measuring the concentration of dissolved oxygen in the sample before and after the step of keeping the sample in the absence of light.

47. The method according to claim 40, wherein more than one steps of measuring the concentration of dissolved oxygen are carried out, and wherein after a first step of measuring the concentration of dissolved oxygen in the sample, the method comprises a step of renewing the sample in the chamber (3) with a new sample from the bioreactor (2) and a second step of measuring the concentration of dissolved oxygen in the sample.

48. The method according to claim 40, comprising more than one steps of illuminating samples, and more than one steps of keeping samples in the absence of light, each step of illuminating a sample alternating with a step of keeping a sample in the absence of light.

49. The method according to claim 40, wherein the culture also comprises bacteria,.

50. A device (1) for characterizing a culture comprising microalgae, the device (1) comprising:
a bioreactor (2) configured to receive a culture comprising microalgae;
a chamber (3) having an inlet (3a) and an outlet (3b), and fluidically connected to the bioreactor (2) so that it can be directly supplied via the inlet (3a) with a sample of culture from the bioreactor (2);
a source for emitting light in the chamber (3); and
at least one sensor (6) for measuring the concentration of dissolved oxygen in the sample.

51. An apparatus for characterizing a culture comprising microalgae, the apparatus comprising:
a chamber (3) having an inlet (3a) and an outlet (3b), the chamber (3) being configured to be fluidically connected to a bioreactor (2) configured to receive a culture comprising microalgae, so that the chamber (3) can be directly supplied via the inlet (3a) with a sample of culture from the bioreactor (2);
a source for emitting light in the chamber (3); and
at least one sensor (6) for measuring the concentration of dissolved oxygen in the sample.

52. The apparatus according to claim 51, wherein the chamber (3) is sealed.

53. The apparatus according to claim 51, comprising at least one additional chamber (4) having an inlet (4a) and an outlet (4b), the additional chamber (4) being fluidically connected to the chamber (3) and the bioreactor (2).

54. The apparatus according to claim 53, comprising a sensor (6) for measuring the concentration of dissolved oxygen located in the chamber (3) and/or comprising a sensor (6) for measuring the concentration of dissolved oxygen located in the additional chamber (4) and/or comprising at least one sensor for measuring the concentration of dissolved carbon dioxide in the sample and/or comprising a sensor (6) for measuring the concentration of dissolved oxygen upstream of the inlet (3a) and/or downstream of the outlet (3b) of the chamber (3); and/or comprising a sensor (6) for measuring the concentration of dissolved oxygen upstream of the inlet (4a) and/or downstream of the outlet (4b) of the additional chamber (4).

55. The apparatus according to claim 53, comprising at least one pump for the circulation of the sample from the bioreactor (2) to the chamber (3) and/or from the chamber (3) to the bioreactor (2), and/or comprising one or more injectors for adding a substrate for microalgae and/or bacteria or an inhibitor or toxic compound for bacteria or microalgae into or upstream of the chamber (3) and/or into or upstream of the additional chamber (4).

56. A computer program product comprising a computer readable medium embodying computer implemented instructions for performing the method of claim 40.

57. A data storage medium having recorded thereon a computer program implementing the method of claim 40.

58. A system comprising a processor coupled to a memory having recorded thereon computer instructions which when executed by the processor performs the method of claim 40.

59. An assembly comprising:
a computer-based system formed of a processor coupled to a memory having recorded thereon computer instructions which when executed by the processor performs the method of claim 40; and
an apparatus for characterizing a culture comprising microalgae, the apparatus comprising:
the chamber (3) having an inlet (3a) and an outlet (3b), the chamber (3) being configured to be fluidically connected to the bioreactor (2) configured to receive a culture comprising microalgae, so that the chamber (3) can be directly supplied via the inlet (3a) with the sample of culture from the bioreactor (2);
a source for emitting light in the chamber (3); and
at least one sensor (6) for measuring the concentration of dissolved oxygen in the sample.
